(19) United States
(12) Patent Application Publication
(10) Pub. No.: US 2010/0119466 A1
(43) Pub. Date: May 13, 2010

(54) TATTOO-REMOVING COMPOSITION

(75) Inventor: Lothar Schibilla, Neumunster (DE)

Correspondence Address: DENNISON, SCHULTZ & MACDONALD, 1727 KING STREET, SUITE 105, ALEXANDRIA, VA, 22314, US

(73) Assignee: SK AGELESS CONCEPT GMBH, Neumunster (DE)

(21) Appl. No.: 12/595,879

(22) PCT Filed: Apr. 15, 2008
(86) PCT No.: PCT/DE2008/000633

§ 371 Date: Oct. 14, 2009

(30) Foreign Application Priority Data
Apr. 19, 2007 (DE) 10 2007 018 886.4
Jul. 12, 2007 (DE) 10 2007 032 844.5

(51) Int. Cl. A61K 8/22; A61B 17/50; A61K 33/40; A61Q 19/00

(52) U.S. Cl. 424/62; 606/131; 424/616

(57) ABSTRACT

A method and a composition for removing a tattoo present on a skin area after prior depilation, disinfection and a treatment to at least partially ablate the epidermis in the area of the tattoo is described, the composition having a liquid to gel-like consistency and comprising a mixture of
a) water,
b) ethyl lactate,
c) 4-hydroxy-2-nonenal (HNE) and/or d-erythro-2-aminooctadec-4-ene-1,3-diol and/or trihydroxypalmitamidohydroxypropyl myristyl ester,
d) 4-hydroxybutyl-propionic acid,
e) 2-hydroxybutyl-propionic acid,
f) 2-trihydroxypurine and
g) hydrogen peroxide.

The present invention relates to two compositions for removing a tattoo present on a skin area having the features of the preambles of patent claims 1 and 9.

Depending on the particular fashion trend or for other identification purposes, tattoos are applied to the human body using different techniques; after a certain period of time, the wearer is oftentimes desirous to remove this tattoo again or replace it with another, newer tattoo.

To create this tattoo, the skin is typically punctured with one or more needles in a selected skin area in order to introduce a dye, which is typically a color pigment, into the skin. During this tattooing process, care has to be taken that in order to produce a long-lasting tattoo, the dye, which can also be several dyes, is deposited in the skin in such a way that this dye is located neither too superficially nor too deeply. In the first case, the dye placed into the skin in such a manner would grow out as a result of the constant renewal of the upper skin layers so that after a certain time period, the tattoo would have unwantedly disappeared; whereas when the dye is introduced into the skin too deeply, bleeding may occur that leads to the dye being washed out. It is therefore necessary that each of the selected dyes or color pigments is positioned in a middle skin layer to produce a long-lasting tattoo.

Additionally, it should also be pointed out that besides the predominantly professionally produced tattoos described above, tattoos are known that are produced with other colored substances, such as for example ash or ink, or result from the contamination of a wound; in the latter possibility, colored dirt particles in particular can deposit in the dermis. Within the scope of the present application, those tattoos that are produced unprofessionally or result unwantedly from wound contamination shall, for the sake of brevity, be combined with the professionally produced tattoos described in the beginning under the term tattoo.

To remove such tattoos, it is known to cut out these tattoos surgically or to dispose of them by peeling off the skin layers or by means of laser treatment.

EP 1 330 199 B for example describes a device for removing a tattoo that essentially aims to mechanically destroy the colored pigments forming the tattoo using needles vibrating with a constant puncturing frequency, so that these mechanically destroyed colored pigments can then additionally be washed out by skin stimulants that are applied to the skin surface. As skin stimulants, EP 1 330 199 B specifies aqueous solutions of lactic acid, sodium hydrazine, common salt, amino acids, small amounts of oxidizing agents optionally being added to these individual skin stimulants.

The known methods described above, in particular the surgical methods or the laser treatment, however, have the disadvantage that more or less noticeable scarring often occurs in the tattooed skin area after removal of the tattoo, so that on the one hand this skin area is aesthetically displeasing and on the other hand is no longer available for the application of new tattoos, which is nowadays frequently desired.

The present invention is based on the object to provide a composition, which allows a scar-free removal of a tattoo.

According to the invention, this object is solved by a composition with the characterizing features of patent claim 1 and/or with the characterizing features of patent claim 9.

The inventive composition for removing a tattoo present on a skin area after prior depilation, disinfection and a treatment to at least partially ablate the epidermis in the area of the tattoo, in particular to partially ablate the horny skin in the area of the tattoo, provides for the inventive composition to have a liquid to gel-like consistency and comprise as active ingredient a mixture of
a) water,
b) ethyl lactate,
c) 4-hydroxy-2-nonenal (HNE) and/or d-erythro-2-aminooctadec-4-ene-1,3-diol and/or trihydroxypalmitamidohydroxypropyl myristyl ester,
d) 4-hydroxybutyl-propionic acid,
e) 2-hydroxybutyl-propionic acid,
f) 2-trihydroxypurine and
g) hydrogen peroxide.

Surprisingly, it was found that the inventive composition described above is superbly suitable to remove tattoos of any type, such as exemplarily described in the introductory part of the present application, in a quick and pain-free manner without there being a danger that ugly scars remain as is the case with surgical methods or laser treatment of prior art. Even with very deep, frequently multi-colored tattoos, these tattoos could be removed, after renewed application of the inventive composition, if necessary, completely and in particular without pain, so that after a healing period of a few days up to four weeks, an objective observer could no longer notice that a tattoo had once been present on the skin area treated with the inventive composition.

The advantages of the inventive composition described above are attributed to the fact that the ingredients necessarily included in the inventive composition apparently exhibit synergistic action to that effect that on the one hand the inventive composition does not cause any allergies, which might explain the painlessness of the application of the inventive composition, and on the other hand inflammation of the skin area treated with the inventive composition does not occur, this inflammation-free application being in particular explained by the inventive composition comprising hydrogen peroxide in appropriate concentrations. When the inventive composition is applied, it can in particular be observed that already after a short period of action, the color pigments of the tattoo are rejected by the skin and are virtually washed out from the inside by the skin's own liquid system, this wash-out process continuing until scab formation starts affecting the wash-out, which begins, however, only after a few hours or a few days. It would therefore also be advantageous to cover the skin area after application of the inventive composition and after the wash-out has started with a system, such as for example of gauze bandages, that on the one hand prevents undesired premature scab formation and on the other hand additionally aids the wash-out process due to a certain capillary action.

In order to accelerate the penetration of the inventive composition into the skin area to be treated that is provided with the tattoo it is desirable that this skin area is first depilated, disinfected and furthermore treated insofar as at least a part of the epidermis and preferably the horny skin is removed; below, in the inventive method, it is explained which preferred treatment methods are used for this.

By varying the concentration of the ingredients that are a mandatory component of the inventive composition and are listed above under items a) to g), on the one hand the speed of the removal of the respective tattoo and on the other hand, depending on the depth and colorfulness of the tattoo, the number of required applications of the inventive composition can be varied. In case of light tattoos, i.e. tattoos characterized by a small area of the tattoo itself and/or by the color depth of the tattoo, those concentration ranges as specified below by the lower concentration value for ingredients b) to g) are principally selected, so that here the water concentration is relatively high; whereas for very color-intensive tattoos and/or for large-area tattoos, those concentrations of ingredients b) to g) as described below by the respective higher concentrations of ingredients b) to g) are preferred.

Considering the aspects given above, the inventive composition hence generally comprises ingredients a) to g) in a preferred concentration range between
a) 2 and 9.5% of water,
b) 24 and 40% of ethyl lactate,
c) 0.18 and 0.55% of 4-hydroxy-2-nonenal (HNE) and/or 2 and 12% of d-erythro-2-aminooctadec-4-ene-1,3-diol and/or 1 and 3% of trihydroxypalmitamidohydroxypropyl myristyl ester,
d) 29 and 39% of 4-hydroxybutyl-propionic acid,
e) 12 and 25% of 2-hydroxybutyl-propionic acid,
f) 0.8 and 5.5% of 2-trihydroxypurine and
g) 2.5 and 17% of hydrogen peroxide.

If the inventive composition comprises a mixture that includes as sole ingredient c) the compound 4-hydroxy-2-nonenal (HNE), a composition has proven particularly advantageous and versatile that comprises with respect to qualitative and quantitative specifications the following ingredients:
a) 4 to 7.5% of water,
b) 28 to 36% of ethyl lactate,
c) 0.2 to 0.5% of 4-hydroxy-2-nonenal (HNE),
d) 31.5 to 37% of 4-hydroxybutyl-propionic acid,
e) 14 to 22.5% of 2-hydroxybutyl-propionic acid,
f) 1 to 5% of 2-trihydroxypurine and
g) 7 to 15% of hydrogen peroxide

If, on the other hand, d-erythro-2-aminooctadec-4-ene-1,3-diol is present as sole ingredient c), an embodiment that is particularly advantageous for this composition comprises ingredients a) to g) in the following concentration ranges; according to experience, in this further embodiment of the inventive composition it is sufficient to appropriately reduce the hydrogen peroxide concentration in comparison to the embodiment of the inventive composition described above here. Hence, this embodiment of the inventive composition comprises the following ingredients
a) 4 to 7.5% of water,
b) 28 to 36% of ethyl lactate,
c) 3 to 10% of d-erythro-2-aminooctadec-4-ene-1,3-diol,
d) 31.5 to 37% of 4-hydroxybutyl-propionic acid,
e) 14 to 22.5% of 2-hydroxybutyl-propionic acid,
f) 1 to 3% of 2-trihydroxypurine and
g) 3.5 to 10% of hydrogen peroxide

A further possibility of varying the concentration of ingredients b) to g) mentioned above of the inventive composition is given by the fact that not the concentrated ingredients themselves but appropriately diluted solutions thereof are mixed together here; ingredient b), i.e. ethyl lactate, in particular being used as a 1 to 3% by weight and preferably as a 1.8% by weight solution, ingredient c), i.e. 4-hydroxybutyl-propionic acid, being used as a 14 to 22% by weight solution and preferably as a 18% by weight solution, ingredient e), i.e. 2-hydroxybutyl-propionic acid, being used as a 7 to 13% by weight and preferably as a 10% by weight solution, and ingredient g), i.e. hydrogen peroxide, being used as a 16 to 24% by weight and preferably as a 20% by weight solution.

The second variant of the inventive composition that is also suitable particularly with regard to the removal of tattoos differs in principle from the embodiments of the first variant of the inventive composition described above in that different ingredients are present here. All other properties described above also apply to the second variant of the inventive composition, as defined in patent claim 9, described below. This variant of the inventive composition comprises the ingredients
a) water,
b) ethyl (S)-2-hydroxypropionate,
c) 2-hydroxypropionic acid,
d) (S)-(−)-butyl lactate and
e) hydrogen peroxide,
   this variant of the inventive composition also having a liquid to gel-like consistency, in particular in the viscosity range as specified above for the first variant of the inventive composition. The pH value of the second variant of the inventive composition in particular also is in the range as described above for the first variant concretized by patent claim 1.

The advantages of this second variant do not differ from the advantages of the first variant of the inventive composition. However, the second variant of the inventive composition has, compared to the first variant of the inventive composition described above, the further advantage that here the number of ingredients is lower, so that hereby the potential danger of the occurrence of allergic skin reactions is further reduced.

By varying the concentration of the ingredients that are a mandatory component of the second variant of the inventive composition and are listed above under items a) to e), on the one hand the speed of the removal of the respective tattoo and on the other hand, depending on the depth and colorfulness of the tattoo, the number of required applications of the second variant of the inventive composition can be varied. In case of light tattoos, i.e. tattoos characterized by a small area of the tattoo itself and/or by the color depth of the tattoo, those concentration ranges as specified below by the lower concentration value for ingredients b) to e) are principally selected, so that here the water concentration is relatively high; whereas for very color-intensive tattoos and/or for large-area tattoos, those concentrations of ingredients b) to e) as described below by the respective higher concentrations of ingredients b) to e) are preferred.

Considering the aspects given above, the inventive composition hence generally comprises in the second variant ingredients a) to e) in a preferred concentration range between
a) 14 and 20% of water,
b) 9 and 15% of ethyl (S)-2-hydroxypropionate,
c) 35 and 44% of 2-hydroxypropionic acid,
d) 5 and 14% of (S)-(−)-butyl lactate and
e) 18 and 28% of hydrogen peroxide,
   and in particular ingredients a) to e) in a particularly preferred concentration range between
a) 16 and 18% of water,
b) 11 and 13% of ethyl (S)-2-hydroxypropionate,
c) 38 and 40% of 2-hydroxypropionic acid,
d) 8 and 10% of (S)-(−)-butyl lactate and
e) 22 and 24% of hydrogen peroxide.

With regard to ethyl (S)-2-hydroxypropionate included in the second variant of the inventive composition it should be noted that this ethyl (S)-2-hydroxypropionate preferably has a concentration between 95 and 99%, whereas the second variant of the inventive composition preferably comprises hydrogen peroxide as a 16 to 24% by weight aqueous solution.

A particularly suitable advancement of the embodiments of the inventive composition described above that in particular has high storage stability, combined with low reduction of the hydrogen peroxide concentration, provides for the ingredients described above to be packaged separately from hydrogen peroxide. It is thereby effectively achieved that even with a quite acidic pH value, decomposition of hydrogen peroxide, in particular at high storage temperatures, cannot occur since hydrogen peroxide is only mixed with the remaining ingredients of the composition immediately prior to application.

Another possibility to stabilize hydrogen peroxide is provided in embodiments of the inventive composition in which the inventive composition comprises besides the ingredients described above in addition at least one stabilizer for hydrogen peroxide and/or at least one complexing agent, the complexing agent, which is in particular ethylenediaminetetraacetic acid and preferably the sodium salt of ethylenediaminetetraacetic acid, ensuring that in particular those metal ions or metals that catalyze the decomposition of hydrogen peroxide are hereby complexed. This, in turn, leads to a further stabilization of the inventive composition in particular during its storage.

Particularly suitable stabilizers provided for in the embodiments described above of the inventive composition are selected from the group comprising phenacetin, phosphoric acid, in particular orthophosphoric acid, α-bisabolol, terpenes, thymol, menthol and camphor and derivatives of the substances mentioned above.

With regard to the concentration of the stabilizers for hydrogen peroxide mentioned above, it should generally be noted that the concentration thereof depends on the desired stabilization and the respective chemical constitution of the stabilizer. In particular, if the inventive composition comprises a stabilizer based on phenacetin or a phenacetin derivative, for example in particular acetaminophen (paracetamole), the concentration of this particular stabilizer in the inventive composition is preferably varied up to 0.5% and most preferably between 0.001% and 0.2%; whereas in case phosphoric acid, in particular orthophosphoric acid and/or phosphoric acid derivatives, is selected, the stabilizer concentration in the inventive composition is in particular up to 3% and preferably between 0.3% and 2%.

Further stabilization, in particular with respect to long-time storage can be achieved with the inventive composition by mixing hydrogen peroxide together with the stabilizers and/or complexing agents described above and packaging it separately from the remaining ingredients of the inventive composition. Accordingly, with this composition that is highly stable in storage, the two separately packaged ingredients of the composition are only mixed with one another immediately prior to application.

For clarification, it should be said that all concentration specifications given in the present description are concretized as percent by weight unless it is explicitly indicated that they represent volume percent. Furthermore, all concentration specifications in percent refer to the ready-to-use composition as it is applied to the concerned skin region to remove the tattoo.

Within the meaning of the present application, water comprises all aqueous systems that are physiologically safe and approved and also includes, besides distilled water, deionized water, high-purity water, those aqueous systems that comprise appropriate buffer systems to correct the pH value or include salts, in particular common salt. Furthermore, it should be noted that salts or derivatives of the ingredients listed above under features b) to g) also fall within the respective ingredients themselves, the term 2-trihydroxypurine in particular also covering 2,6,8-trihydroxypurine and the term trihydroxypalmit-amidohydroxypropyl myristyl ester also covering the compound trihydroxypalmitamidohydroxypropyl myristyl ether. The term “and/or” used in the present text means that the individual elements in the respective list are to be viewed both additively and alternatively; in the additive naming, at least two elements of the list are combined with one another.

Preferably, the inventive composition has a pH value that is in the range of pH 7, this range covering in particular a pH value between 5.3 and 7.9.

In order to guarantee a gel-like consistency of the inventive composition it would be advantageous to provide for the customary gelling agents used in the cosmetic sector so that the viscosity, measured at 20° C., of these embodiments of the inventive composition preferably varies between 5 mPas and 40,000 mPas and in particular between 100 mPas and 3,000 mPas. If required, the inventive composition can further include a preservative and the additives customary in the cosmetic sector.

In addition, the present invention relates to a method for removing a tattoo, the inventive method providing for the skin area provided with the tattoo, after depilation and/or disinfection, to be treated in such a way that at least part of the epidermis and preferably the top part of the epidermis and hence also the top part of the horny skin (stratum corneum) is removed and afterwards a composition, as described above as inventive composition, is applied to the skin area pre-treated in such a manner, the inventive composition being allowed to take effect for a predetermined time period.

The inventive method has identically or analogously all advantages as described in detail above for the two variants of the inventive compositions. However, regarding the inventive method it should be noted in particular that the inventive method is particularly easy to apply; application of the inventive method does not necessarily require the action of a doctor or dermatologist but can also be done by appropriately trained personnel such as for example tattoo artists or cosmeticians since the inventive method is not a surgical or therapeutic and hence not a medicinal method.

In order to facilitate the penetration of the inventive composition into the respective skin area to be treated that is provided with the tattoo, as briefly discussed above in connection with the inventive composition, a first advancement of the inventive method provides for the skin area to be mechanically treated spot by spot and/or over small areas. Spot-by-spot treatment of this skin area through which the top region of the epidermis is at least partially perforated is present if an implement, for example a needle or a suitable rotating fraise tip, is used for this mechanical treatment, the size of an area produced hereby being less than 2 mm², so that the epidermis in its outer region is correspondingly perforated spot by spot with very small holes. If a small-area treatment of the skin area is performed, it is advantageous for this to vary the size of each area produced hereby between 2 mm² and 15 mm², the distance between adjacent areas then being in particular chosen in such a way that this distance is here between 2 mm and 15 mm.

Surprisingly, when applying the inventive method it is sufficient if the respective mechanical treatment is done up to a depth between 0.05 mm and 0.2 mm, the depth naturally depending upon on which body region each tattoo to be removed was positioned. Accordingly, with such a small depth, only the horny skin (stratum corneum) is appropriately partially perforated in the inventive method, the inventive composition quickly penetrating to the actual tattoo via these perforations and there effecting the wash-out process addressed above.

In particular, abrasion with a fraise of the skin area is performed as mechanical treatment in the inventive method, high-speed fraises preferably being used here that are equipped with a diamond or blue diamond fraise tip, the fraise tip in particular rotating with a rotational speed between 220,000 rpm to 270,000 rpm.

Another possibility to treat the skin area in the inventive method is provided by sanding the whole skin area provided with the tattoo, suitable abrasives such as for example abrasive cloths or abrasive pastes, typically referred to as peeling pastes, being used for this. The depth of this abrading preferably varies between 0.05 mm and 0.2 mm so that even when the whole area is abraded, scarring is eliminated.

Another possibility to facilitate and accelerate the penetration of the inventive compositions in the inventive method results when the skin area provided with the tattoo is superficially provided with microcuts, i.e. hence linearly, the depth of such microcuts preferably varying between 0.05 mm and 0.2 mm. It is also possible in the inventive method to chemically treat, either spot by spot, linearly, over small areas or in particular over the whole area, the skin area having the tattoo that is to be removed, the upper region of the horny skin in particular being swelled or removed by such a chemical treatment. For this, for example alkaline solutions, in particular alkaline aqueous solutions and/or surfactant solutions can be used; this process can additionally be accelerated by employing an occlusive dressing technique to remove this skin area.

Another possibility to treat the skin area provided with the tattoo results when a thermal treatment, in particular a thermal treatment using a needle heated by a high-frequency current, is provided for in the inventive method in order to hereby remove the outer skin area up to a depth of preferably between 0.05 mm and 0.2 mm and hence facilitate the penetration of the inventive compositions into the tattoo area and the wash-out of the dyes or colored pigments.

With regard to the predetermined time period specified in the inventive method, which follows the penetration of the inventive compositions into the tattoo area and the subsequent wash-out, it should generally be noted that this time period is adjusted on the one hand to the depth of the tattoo and on the other hand to the colorfulness of the tattoo and in addition depends on the size of the area covered by the tattoo. Surprisingly, it was found that in particular a time period between 5 minutes and 60 minutes is chosen to already achieve a good effect with regard to the removal of the tattoo; after this time period in which the inventive compositions are re-applied, if necessary, has expired, excess inventive compositions remaining on the skin area is washed off and afterwards the skin area treated by the inventive method is dressed, the inventive method possibly being repeated several times after a preferred interval of three to six weeks each, if necessary.

Advantageous embodiments of the inventive compositions and the inventive method are given in the sub-claims. Below, the inventive compositions are explained in more detail by means of three examples.

### EXAMPLE 1

A Composition I was prepared, which comprised the following ingredients, all concentrations being specified in percent by weight:
a) 6.5% of water, ultrapure DAB7
b) 31% of ethyl lactate,
c) 0.5% of 4-hydroxy-2-nonenal (HNE),
d) 28% of 4-hydroxybutyl-propionic acid,
e) 20% of 2-hydroxybutyl-propionic acid,
f) 2% of 2-trihydroxypurine and
g) 12% of hydrogen peroxide.

Ethyl lactate was a 1.8% solution, 4-hydroxybutyl-propionic acid was an 18% solution, 2-hydroxybutyl-propionic acid was a 10% solution, whereas hydrogen peroxide was a 20% aqueous solution.

For the preparation of Composition I, the components listed above were mixed using a high-speed stirring device (1,000 rpm) at room temperature until a homogeneous liquid mixture having a watery consistency had formed.

Composition I prepared in this manner did not show any demixing even after a storage time of 12 weeks.

A female proband, who had on the right upper arm a tattoo that depicted a salamander and was approximately 10 cm long and that primarily comprised the colors blue and red, was treated, after prior shaving of the hair and disinfection of the skin area provided with the tattoo, with Composition I as follows:

First, the skin covering the tattoo was abraded spot by spot using a high-speed fraise with a rotating diamond fraise tip, the fraise having a rotational speed of 250,000 revolutions/min (rpm) for this purpose. The distance of the holes produced by the fraise of a size of approximately 1 mm² in the skin area located above the tattoo was 10 mm all-around so that a symmetrical pattern of holes was formed. The depth was set to 0.15 mm.

Composition I was applied to the skin area treated in this manner; care was taken that the treated skin area was always kept moist with Composition I. After a dwell time, i.e. a predetermined time period of 50 minutes, and after color pigments had already been washed out, the skin area was washed with sterile water and afterwards covered with a gauze bandage that was repeatedly changed over the next three days. It was found hereby that color pigments were permanently washed out through the holes produced by the fraise.

After three days had expired, the tattoo treated in this manner was already noticeably reduced in surface area and contour, i.e. areas in which color pigments were no longer present already appeared.

The treatment described above was repeated in identical manner after three weeks and after six weeks, each calculated starting from the first treatment.

After eight weeks, calculated from the beginning of treatment, five objective experts were instructed to locate the skin area previously tattooed on the arm of the female proband. None of the five experts was able to correctly determine the previously tattooed area. Scars or other changes of the skin in the area previously tattooed could also not be found.

Neither during treatment nor afterwards the female proband reported any pain. Inflammation of the treated skin area also did not occur.

### EXAMPLE 2

A Composition II was prepared, which comprised the following ingredients, all concentrations being specified in percent by weight:
a) 5.9% of water, ultrapure DAB 7
b) 29.5% of ethyl lactate,
c) 3.5% of d-erythro-2-aminooctadec-4-ene-1,3-diol,
d) 34.3% of 4-hydroxybutyl-propionic acid,
e) 14.5% of 2-hydroxybutyl-propionic acid,
f) 2.3% of 2-trihydroxypurine and
g) 10% of hydrogen peroxide.

Ethyl lactate was a 1.8% solution, 4-hydroxybutyl-propionic acid was an 18% solution, 2-hydroxybutyl-propionic acid was a 10% solution, whereas hydrogen peroxide was a 20% aqueous solution.

For the preparation of Composition II, the components listed above were mixed using a high-speed stirring device (1,000 rpm) at room temperature until a homogeneous liquid mixture having a watery consistency had formed.

Composition II prepared in this manner did not show any demixing even after a storage time of two month.

A male proband, who had on the left forearm a tattoo that was approximately 8 cm long and approximately 4 cm wide and depicted a heart and an arrow penetrating this heart and that comprised primarily the color black was treated, after prior shaving of the hair and disinfection of the skin area provided with the tattoo, with Composition II as follows:

Using a commercially available epilator of type Danycare, T-Away, by means of a needle that was permanently heated by a high-frequency current, the skin in the skin area covering the tattoo was linearly (width approx. 2 mm) ablated layer by layer; the depth of ablation was approximately 0.15 mm.

Composition II was applied to the skin area treated in this manner; care was taken that the treated skin area was always kept moist with Composition II. After a dwell time of 30 minutes and after color pigments had already been washed out, the skin area was washed with sterile water and afterwards covered with a gauze bandage that was repeatedly changed over the next two days. It was found hereby that color pigments were permanently washed out through the linear depressions.

After two days had expired, the tattoo treated in this manner was already noticeably reduced in surface area and contour, i.e. areas in which color pigments were no longer present already appeared.

The treatment described above was repeated in identical manner after two weeks and after four weeks, each calculated starting from the first treatment.

After six weeks, calculated from the beginning of treatment, three objective experts were instructed to locate the skin area previously tattooed on the arm of the male proband. None of the three experts was able to correctly determine the previously tattooed area. Scars or other changes of the skin in the area previously tattooed could also not be found.

Neither during treatment nor afterwards the male proband reported any pain. Inflammation of the treated skin area also did not occur.

### EXAMPLE 3

A Composition III was prepared, which comprised the following ingredients, all concentrations being specified in percent by weight:
a) 17% of water,
b) 12% of ethyl (S)-2-hydroxypropionate,
c) 39% of 2-hydroxypropionic acid,
d) 9% of (S)-(−)-butyl lactate and
e) 23% of hydrogen peroxide.

Ethyl (S)-2-hydroxypropionate was present in a concentration of 98% by weight, whereas hydrogen peroxide was a 20% aqueous solution.

For the preparation of Composition III, the components listed above were mixed using a high-speed stirring device (1,000 rpm) at room temperature until a homogeneous liquid mixture having a watery consistency had formed.

Composition III prepared in this manner did not show any demixing even after a storage time of two month.

A male proband, who had on the left upper arm a garland-like tattoo that was approximately 12 cm long and approximately 3 cm wide and primarily comprised the color black was treated, after prior shaving of the hair and disinfection of the skin area provided with the tattoo, with Composition III as follows:

Using a commercially available epilator of type Danycare, T-Away, by means of a needle that was permanently heated by a high-frequency current, the skin in the skin area covering the tattoo was linearly (width approx. 2 mm) ablated layer by layer; the depth of ablation was approximately 0.15 mm.

Composition III was applied to the skin area treated in this manner; care was taken that the treated skin area was always kept moist with Composition III. After a dwell time of 30 minutes and after color pigments had already been washed out, the skin area was washed with sterile water and afterwards covered with a gauze bandage that was repeatedly changed over the next three days. It was found hereby that color pigments were permanently washed out through the linear depressions.

After three days had expired, the tattoo treated in this manner was already noticeably reduced in surface area and contour, i.e. areas in which color pigments were no longer present already appeared.

The treatment described above was repeated in identical manner after two and a half weeks and after five weeks, each calculated starting from the first treatment.

After seven weeks, calculated from the beginning of treatment, three objective experts were instructed to locate the skin area previously tattooed on the upper arm of the male proband. None of the three experts was able to correctly determine the previously tattooed area. Scars or other changes of the skin in the area previously tattooed could also not be found.

Neither during treatment nor afterwards the male proband reported any pain. Inflammation or an allergic reaction of the treated skin area also did not occur.

1. A composition for removing a tattoo present on a skin area after prior depilation, disinfection and a treatment to at least partially ablate the epidermis in the area of the tattoo, characterized in that said composition has a liquid to gel-like consistency and comprises a mixture of
a) water,
b) ethyl lactate,
c) 4-hydroxy-2-nonenal (HNE) and/or d-erythro-2-aminooctadec-4-ene-1,3-diol and/or trihydroxypalmitamidohydroxypropyl myristyl ester,
d) 4-hydroxybutyl-propionic acid,
e) 2-hydroxybutyl-propionic acid,
f) 2-trihydroxypurine and
g) hydrogen peroxide.

2. The composition according to claim 1, characterized in that said composition comprises between
a) 2 and 9.5% of water,
b) 24 and 40% of ethyl lactate,
c) 0.18 and 0.55% of 4-hydroxy-2-nonenal (HNE) and/or 2 and 12% of d-erythro-2-aminooctadec-4-ene-1,3-diol and/or 1 and 3% of trihydroxypalmitamidohydroxypropyl myristyl ester,
d) 29 and 39% of 4-hydroxybutyl-propionic acid,
e) 12 and 25% of 2-hydroxybutyl-propionic acid,
f) 0.8 and 5.5% of 2-trihydroxypurine and
g) 2.5 and 17% of hydrogen peroxide.

3. The composition according to claim 1, characterized in that said composition comprises a mixture of
a) 4 to 7.5% of water,
b) 28 to 36% of ethyl lactate,
c) 0.2 to 0.5% of 4-hydroxy-2-nonenal (HNE),
d) 31.5 to 37% of 4-hydroxybutyl-propionic acid,
e) 14 to 22.5% of 2-hydroxybutyl-propionic acid,
f) 1 to 5% of 2-trihydroxypurine and
g) 7 to 15% of hydrogen peroxide.

4. The composition according to claim 1, characterized in that said composition comprises a mixture of
a) 4 to 7.5% of water,
b) 28 to 36% of ethyl lactate,
c) 3 to 10% of d-erythro-2-aminooctadec-4-ene-1,3-diol,
d) 31.5 to 37% of 4-hydroxybutyl-propionic acid,
e) 14 to 22.5% of 2-hydroxybutyl-propionic acid,
f) 1 to 3% of 2-trihydroxypurine and
g) 3.5 to 10% of hydrogen peroxide.

5. The composition according claim 1, characterized in that said composition comprises ethyl lactate as a 1 to 3% by weight solution.

6. The composition according claim 1, characterized in that said composition comprises 4-hydroxybutyl-propionic acid as a 14 to 22% by weight solution.

7. The composition according claim 1, characterized in that said composition comprises 2-hydroxybutyl-propionic acid as a 7 to 13% by weight solution.

8. The composition according claim 1, characterized in that said composition comprises hydrogen peroxide as a 16 to 24% by weight solution.

9. A composition for removing a tattoo present on a skin area after prior depilation, disinfection and a treatment to at least partially ablate the epidermis in the area of the tattoo, characterized in that said composition has a liquid to gel-like consistency and comprises a mixture of
a) water,
b) ethyl (S)-2-hydroxypropionate,
c) 2-hydroxypropionic acid,
d) (S)-(−)-butyl lactate and
e) hydrogen peroxide.

10. The composition according to claim 9, characterized in that said composition comprises between
a) 14 and 20% of water,
b) 9 and 15% of ethyl (S)-2-hydroxypropionate,
c) 35 and 44% of 2-hydroxypropionic acid,
d) 5 and 14% of (S)-(−)-butyl lactate and
e) 18 and 28% of hydrogen peroxide.

11. The composition according to claim 9, characterized in that said composition comprises between
a) 16 and 18% of water,
b) 11 and 13% of ethyl (S)-2-hydroxypropionate,
c) 38 and 40% of 2-hydroxypropionic acid,
d) 8 and 10% of (S)-(−)-butyl lactate and
e) 22 and 24% of hydrogen peroxide.

12. The composition according to claim 9, characterized in that the ethyl (S)-2-hydroxypropionate included in the composition has a concentration between 95 and 99%.

13. The composition according to claim 9, characterized in that said composition comprises hydrogen peroxide as a 16 to 24% by weight solution.

14. The composition according claim 9, characterized in that hydrogen peroxide is packaged separately from the remaining ingredients of the composition.

15. The composition according claim 9, characterized in that said composition comprises at least one stabilizer for the hydrogen peroxide and/or at least one complexing agent.

16. The composition according to claim 15, characterized in that the stabilizer for the hydrogen peroxide is selected from the group comprising phenacetin, phosphoric acid, a-bisabolol, terpenes, thymol, menthol and camphor and derivatives of the substances mentioned above.

17. The composition according to claim 15, characterized in that said composition comprises phenacetin in a concentration of up to 0.5%, in particular between 0.001% and 0.2%, and/or phosphoric acid in a concentration of up to 3%, in particular between 0.3% and 2%.

18. A method for removing a tattoo wherein the skin area provided with the tattoo, after depilation and/or disinfection, is treated in such a way that at least part of the epidermis is removed, and afterwards a composition according to claim 1 is applied to the skin area and allowed to take effect for a predetermined time period.

19. The method according to claim 18, characterized in that treatment of the skin area is done mechanically spot by spot and/or over small areas.

20. The method according to claim 18, characterized in that the mechanical treatment of the skin area is done over small areas, the size of each area varying between 2 mm² and 15 mm².

21. The method according to claim 18, characterized in that the distance between adjacent areas varies between 2 mm and 15 mm.

22. The method according to claim 18, characterized in that the mechanical treatment is done up to a depth between 0.05 mm and 0.2 mm.

23. The method according to claim 18, characterized in that the mechanical treatment is done by abrasion of the skin area using a fraise.

24. The method according to claim 18, characterized in that as predetermined time period, a time period between 5 minutes and 60 minutes is selected.

25. The method according to claim 18, characterized in that the treatment comprises thermal treatment.

26. The method according to claim 18, characterized in that the whole skin area is treated mechanically and/or chemically.

27. The method according to claim 26, characterized in that the skin area is sanded.
